(19) United States
(12) Patent Application Publication
(10) Pub. No.: US 2024/0140950 A1
(43) Pub. Date: May 2, 2024

(54) NITROGEN-CONTAINING POLYCYCLIC AROMATIC COMPOUND, and PREPARATION METHOD AND APPLICATION THEREOF

(71) Applicant: Hebei University of Science & Technology, Hebei (CN)

(72) Inventor: Huajie ZHU, Hebei (CN)

(73) Assignee: Hebei University of Science & Technology, Hebei (CN)

(21) Appl. No.: 18/398,201

(22) Filed: Dec. 28, 2023

Related U.S. Application Data
(63) Continuation of application No. PCT/CN2022/101788, filed on Jun. 28, 2022.

(30) Foreign Application Priority Data
Jun. 28, 2021 (CN) 202110721322.5

(51) Int. Cl. C07D 471/22; A61P 35/00

(52) U.S. Cl. CPC C07D 471/22; A61P 35/00

(57) ABSTRACT

The present application provides a nitrogen-containing polycyclic aromatic compound and its preparation method and application. The nitrogen-containing polycyclic aromatic compound has a structure of Formula I, has an excellent broad-spectrum anti-tumor activity, and shows an inhibitory activity comparable to cisplatin on cervical cancer cells, breast cancer cells, melanoma cells, lung cancer cells and myeloma cells. Compared with cisplatin, the compound also has lower toxicity, and may be used as a lead compound for the development of new anti-tumor drugs.

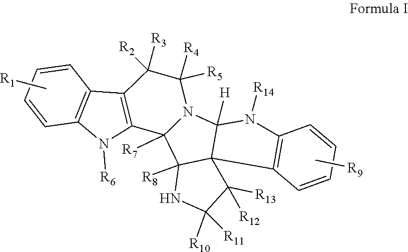

### CROSS-REFERENCE TO RELATED APPLICATIONS

This application is a continuation of International Application No. PCT/CN2022/101788, filed on Jun. 28, 2022, which claims priority to Chinese Patent Application No. 202110721322.5, entitled with “NITROGEN-CONTAINING POLYCYCLIC AROMATIC COMPOUND, AND PREPARATION METHOD AND APPLICATION THEREOF”, and filed with the China National Intellectual Property Administration on Jun. 28, 2021. The disclosures of the aforementioned applications are hereby incorporated by reference in their entireties.

### BACKGROUND

### Technical Field

The present application relates to the field of medicine, and in particular, to a nitrogen-containing polycyclic aromatic compound and its preparation method and application.

### Description of Related Art

Malignant tumor is an abnormality in structure, function and metabolism that leads to abnormal proliferation of local tissues caused by malignant changes of cells. At present, the methods of treating malignant tumor mainly include surgical treatment and radiotherapy/chemotherapy. At least based on current knowledge, tumor diseases and patients suitable for surgical treatment and radiotherapy/chemotherapy are limited by many factors, especially for patients with advanced tumors, there is no effective treatment and mitigation means, so screening and developing new or potential therapeutic drugs has always been the goal of efforts.

Taking cisplatin as an example, it is a non-specific drug for cells. Studies have shown that cisplatin can bind to DNA and cause cross-linking, thereby destroying the function of DNA and inhibiting DNA replication of cells. In clinical application, cisplatin has a broad anti-tumor spectrum, and is applied to head and neck squamous cell carcinoma, ovarian cancer, embryonal carcinoma, seminoma, lung cancer, thyroid cancer, lymphosarcoma and reticulocyte sarcoma, etc. Big data statistics show that it has good tumor treatment effects and is currently considered as one of the most effective broad-spectrum anti-tumor drugs in clinical treatment. However, it also shows serious toxic side effects in clinical practice. For example, cisplatin may cause renal tubular cell death and renal tissue damage through mechanisms such as oxidative stress, DNA damage, and inflammatory response, thereby reducing glomerular filtration rate and significantly reducing renal excretion, resulting in a large amount of cisplatin accumulation in the kidney and thus severe renal failure. Cisplatin may also damage cochlear hair cells, causing hearing loss and leading to deafness or tinnitus. On the other hand, the problem of the resistance to drugs in use has always been a problem that needs to be faced and solved. The corresponding development of upgraded drugs and alternative drugs is always full of expectations for both drug developers and patients.

Therefore, it is of great significance to develop a new broad-spectrum antitumor drug.

### SUMMARY

The present application provides a nitrogen-containing polycyclic aromatic compound, its preparation method and application. The nitrogen-containing polycyclic aromatic compound provided by the present application has excellent broad-spectrum anti-tumor activity and shows low toxicity, and may be used as a lead compound for the development of new anti-tumor drugs.

The present application provides a nitrogen-containing polycyclic aromatic compound and a pharmaceutically acceptable salt thereof, the compound having a structure shown in Formula I:

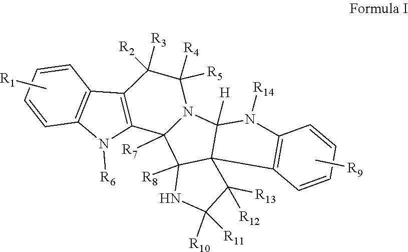

where, R₁ and R₉ are each independently selected from a group consisting of: H, C₁₋₆ alkyl, C₁₋₆ alkoxy, benzyloxy, and halogen;
R₂, R₃, R₄, R₅, R₁₀, R₁₁, R₁₂, and R₁₃ are each independently selected from a group consisting of: H, C₁₋₆ alkyl, —COORₓ; Rₓ is selected from C₁₋₆ alkyl;
R₆ and R₁₄ are each independently selected from: H or C₁₋₆ alkyl;
R₇ and R₈ are each independently selected from H or C₁₋₆ alkyl.

In the present application, the C₁₋₆ alkyl refers to a linear or branched alkyl with 1 to 6 carbon atoms, such as methyl, ethyl, n-propyl, isopropyl, n-butyl, isobutyl, tert-butyl, n-pentyl, isopentyl, neopentyl, n-hexyl, etc.; the C₁₋₆ alkoxy refers to a linear or branched alkoxy with 1 to 6 carbon atoms, such as methoxy, ethoxy, n-propoxy, isopropoxy, n-butoxy, isobutoxy, tert-butoxy, n-pentyloxy, isopentyloxy, neopentyloxy, n-hexyloxy, etc.

According to the nitrogen-containing polycyclic aromatic compound of the present application, the pharmaceutically acceptable salt refers to a salt formed by a suitable non-toxic organic acid, inorganic acid, organic base or inorganic base and a compound, which retains the biological activity of the compound. The specific product of the pharmaceutically acceptable salt may be determined by conventional means in accordance with the well-known knowledge in the field of pharmaceutical research and preparation. Examples are given below.

The organic acid may be selected from the organic acids which are capable of forming salts and are commonly used in the field of pharmaceutical, such as formic acid, acetic acid, propionic acid, trifluoroacetic acid, oxalic acid, benzoic acid, p-toluenesulfonic acid, maleic acid, fumaric acid, citric acid, tartaric acid, malic acid, lactic acid, salicylic acid, and the like.

The inorganic acid may be selected from inorganic acids which are capable of forming salts and are commonly used in the field of pharmaceutical, such as hydrochloric acid, sulfuric acid, phosphoric acid, and the like.

The organic base may be selected from organic bases which are capable of forming salts and are commonly used in the field of pharmaceutical, such as pyridine, imidazole, pyrazine, indole, purine, aniline, and the like.

The inorganic base may be selected from inorganic bases which are capable of forming salts and are commonly used in the field of pharmaceutical, such as alkali metal hydride, alkali metal hydroxide, alkali metal alkoxide, potassium carbonate, sodium carbonate, lithium carbonate, potassium bicarbonate, sodium bicarbonate, and the like. Where, the alkali metal hydride may be sodium hydride and/or potassium hydride; the alkali metal hydroxide may be sodium hydroxide, potassium hydroxide, lithium hydroxide, etc.; and the alkali metal alkoxide may be sodium methoxide, sodium ethoxide, potassium tert-butoxide, sodium tert-butoxide, etc.

Further, the C₁₋₆ alkyl is selected from C₁₋₃ alkyl, such as methyl, ethyl, propyl, isopropyl, and the like; the C₁₋₆ alkoxy is selected from C₁₋₃ alkoxy, such as methoxy, ethoxy, propoxy, isopropoxy, and the like.

In a specific implementation, R₁ and R₉ are each independently selected from one of H, methyl, ethyl, ethoxy, benzyloxy, and chlorine; and/or,
R₂, R₃, R₄, R₅, R₁₀, R₁₁, R₁₂, and R₁₃ are each independently selected from one of H, methyl, ethyl, and —COOMe; and/or,
R₆ and R₁₄ are each independently selected from a group consisting of H, methyl and ethyl; and/or,
R₇ and R₈ are each independently selected from a group consisting of H, methyl and ethyl.

In a specific implementation, R₂, R₃, R₄, R₅, R₁₀, R₁₁, R₁₂, and R₁₃ are each selected from H; or,
R₂, R₃, R₁₂, and R₁₃ are each selected from H, and R₄, R₅, R₁₀, and R₁₁ are each selected from methyl or ethyl; or,
R₂, R₃, R₁₂, and R₁₃ are each selected from methyl, and R₄, R₅, R₁₀, and R₁₁ are each selected from H; or,
R₂, R₃, R₁₂, and R₁₃ are each selected from H, R₄ and R₅ are each selected from H and —COOMe, and R₁₀ and R₁₁ are each selected from H and —COOMe.

In a specific implementation, R₇ and R₈ are both selected from H or methyl; or,
R₇ is selected from methyl, and R₈ is selected from H; or,
R₇ is selected from ethyl, and R₈ is selected from methyl.

In a specific implementation, R₁ and R₉ are each selected from of one of H, methyl, ethyl, ethoxy, benzyloxy and chlorine; and/or,
R₆ and R₁₄ are each selected from H, methyl or ethyl.

In the common knowledge in the field of organic chemistry, the atomic numbers of the indole ring are as follows:

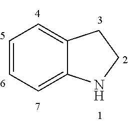

in a possible implementation, R₁ and R₉ are each located at the position where the atomic number of the indole ring is 5.

As a non-limiting example, the nitrogen-containing polycyclic aromatic compound of the present application may be compounds numbered 1 to 22:

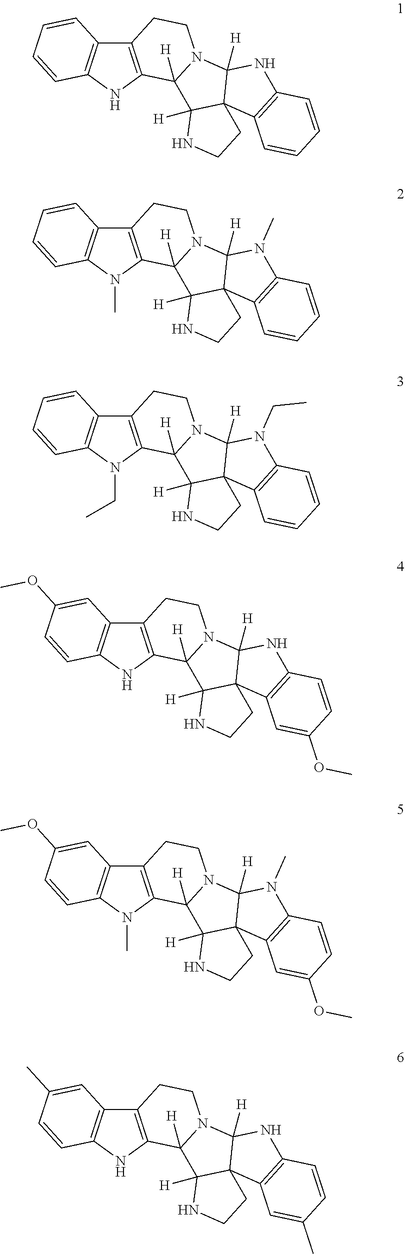

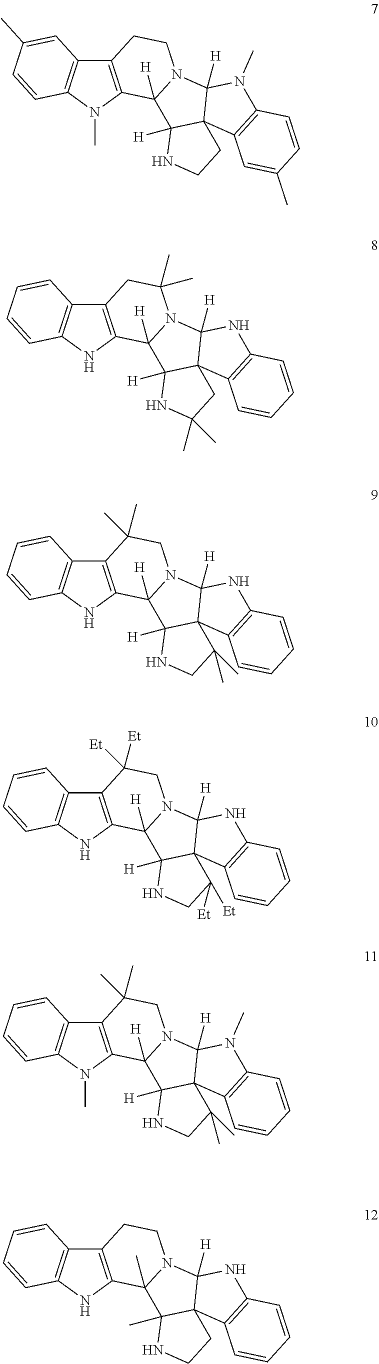

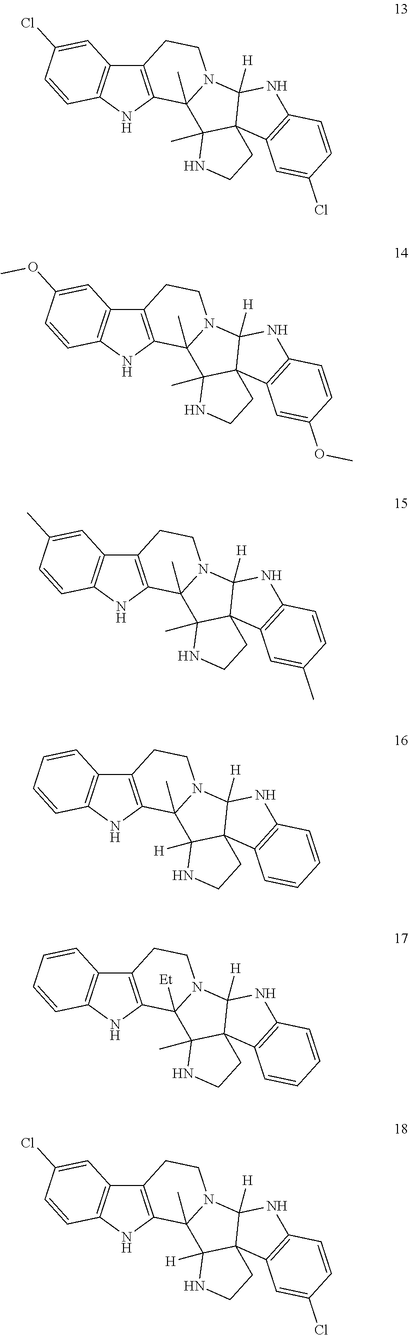

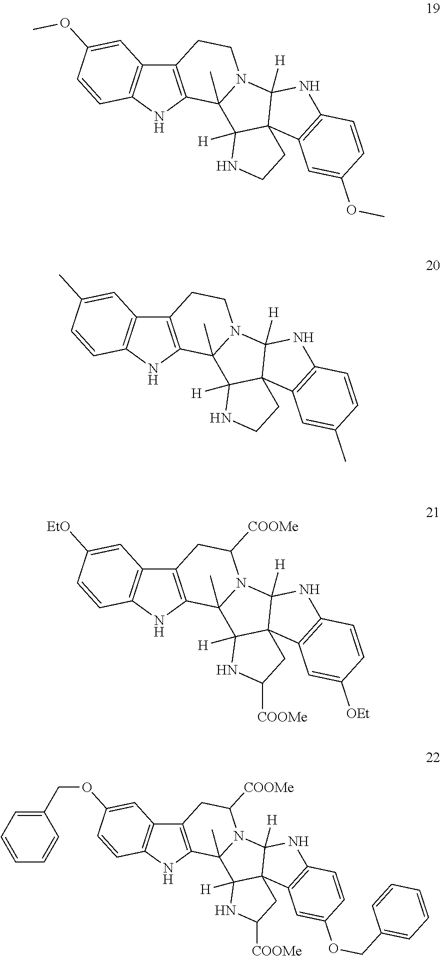

The inventor's research found that a small molecular compound with the structure of Formula I shows excellent broad-spectrum anti-tumor activity. Especially, it shows an inhibitory rate comparable to cisplatin for cervical cancer cells HELA, breast cancer cells MCF-7, MDA-MB-231, melanoma cells A375, lung cancer cells A549 and bone marrow cancer cells SP2/0. It can be seen from its toxicity test on normal cells 293T and the acute toxicity test in mice that the toxicity of the small molecular compound with the structure of Formula I is much lower than that of cisplatin. Therefore, the compound represented by Formula I may be used as a lead compound for the research of new low-toxicity and high-efficiency broad-spectrum anti-tumor drugs.

The present application also provides a preparation method of the aforementioned nitrogen-containing polycyclic aromatic compound, comprising the following steps:

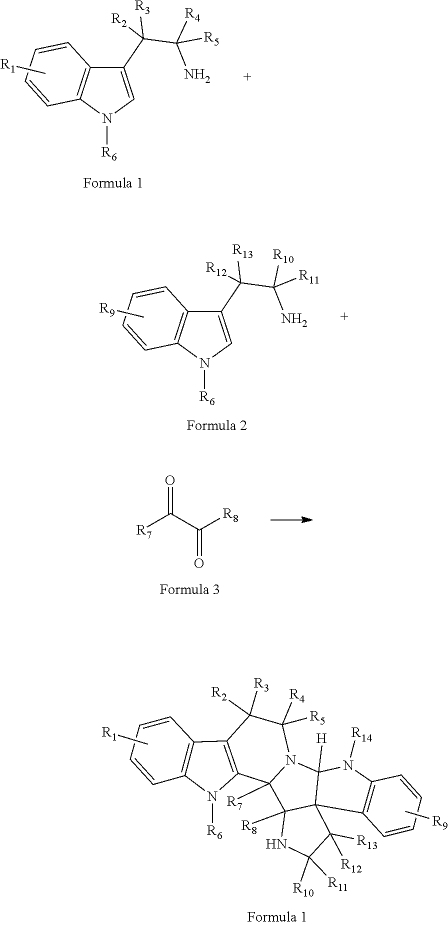

the compound of Formula 1, the compound of Formula 2 and the compound of Formula 3 are reacted under the action of an acid catalyst to obtain the compound represented by Formula I.

The reactants in the above process may be purchased commercially, or synthesized by well-known means, such as referring to Journal of Medicinal Chemistry (2010), 53(14), 5155-5164, CN104529865B and other documents.

In the specific reaction process, in order to ensure that the reaction is performed fully to obtain the target product, the compound of Formula 3 as a reactant may be properly controlled to be excess. Under comprehensive consideration, for example, a molar ratio of the compound of Formula 1, the compound of Formula 2 and the compound of Formula 3 may be controlled to be substantially 1:1:(1-1.4).

The preparation process ends with the reaction of all reactants of compound of Formula 1 and compound of Formula 2 as far as possible to produce the target product. According to the situation of the reactants and the corresponding reaction conditions, it may substantially be determined by proper exploration or by means of conventional means. In the specific operations, for example, by means of TLC (Thin-Layer Chromatography), HPLC (High Performance Liquid Chromatography), NMR (Nuclear Magnetic Resonance), etc., it may be determined that the reaction is completed when the raw materials compound of Formula 1 and compound of Formula 2 in the reaction system disappear substantially.

The preparation conditions of the compound of Formula I are relatively mild, and the products may be obtained by carrying out the above reaction at a temperature of −20° C. to 60° C.

In the solvent system of the above reaction, all of chlorinated solvents, alcohol solvents, and ether solvents can make the reaction proceed smoothly. Exemplarily, the chlorinated solvent may be dichloromethane, chloroform, etc.; the alcohol solvent may be methanol, ethanol, etc.; and the ether solvent may be tetrahydrofuran.

After screening the solvents, the inventors found that when the solvent is selected from dichloromethane, the compound represented by Formula I may be prepared by the reaction with a higher yield.

As aforementioned preparation method, the reaction process is completed under the action of an acid catalyst, and the selection of the specific acid catalyst and reaction conditions is a conventional means for those skilled in the art that have basic knowledge of the synthesis of relevant organic compounds.

For example, the acid catalyst may be selected from acid catalysts which are commonly used in PS (Pseudohalide) reactions, including but not limited to trifluoroacetic acid, glacial acetic acid, hydrochloric acid, p-toluenesulfonic acid, and the like.

The present application also provides a pharmaceutical composition including the above-mentioned nitrogen-containing polycyclic aromatic compound. The pharmaceutical composition of the present application refers to the one which takes the compound represented by Formula 1 as the active ingredient and is supplemented with pharmaceutically acceptable pharmaceutical excipients or carriers.

The pharmaceutical composition of the present application may be prepared into various dosage forms, such as oral formulations, injection formulations, suppositories, etc., by conventional methods in the art. Formulations suitable for oral administration include solid formulations, solutions, suspensions or emulsions, etc., and the solid formulation may specifically be tablets, granules, capsules, powders, etc. Suppositories, for example, pharmaceutical preparations suitable for parenteral administration, injection formulations, for example, which may be intramuscular injections or intravenous drip preparations, etc., may also be made into powder injections or freeze-dried powder injections if necessary.

As pharmaceutical excipients, those conventionally used in the pharmaceutical field may be used. Examples of usable pharmaceutical excipients include an excipient (e.g. carbohydrate derivatives such as lactose, sucrose, glucose, mannitol and sorbitol; starch derivatives such as corn starch, potato starch, dextrin and carboxymethyl starch; cellulose derivatives such as crystalline cellulose, hydroxypropyl cellulose, carboxymethyl cellulose, carboxymethyl cellulose calcium, carboxymethyl cellulose sodium; arabic gum; dextran; silicate derivatives such as magnesium aluminum metasilicate; phosphate derivatives such as calcium phosphate; sulfate derivatives such as calcium sulfate, etc.), a binder (e.g. gelatin, polyvinylpyrrolidone and polyethylene glycol), a disintegrant (e.g. cellulose derivatives such as carboxymethyl cellulose sodium, polyvinylpyrrolidone), a lubricant (e.g. talc, calcium stearate, magnesium stearate, spermaceti, boric acid, sodium benzoate, leucine), a stabilizer (methyl p-hydroxybenzoate, propyl p-hydroxybenzoate, etc.), a flavoring agent (e.g. commonly used sweeteners, sour agents, and spices, etc.), a diluent, and a solvent for injection (such as water, ethanol, and glycerin, etc.).

As mentioned above, the nitrogen-containing polycyclic aromatic compound provided by the present application has excellent broad-spectrum anti-tumor activity, therefore, the present application also provides the use of the above-mentioned nitrogen-containing polycyclic aromatic compound and the pharmaceutically acceptable salt as active ingredients in the preparation of an anti-tumor drug.

Pharmacodynamic studies show that the compound represented by Formula I and the pharmaceutically acceptable salt all exhibit inhibitory activity comparable to cisplatin on cervical cancer cells, breast cancer cells, melanoma cells, lung cancer cells, and bone marrow cancer cells.

The present application also provides a method for preventing and/or treating a tumor, the method comprising: administering a drug containing the aforementioned nitrogen-containing polycyclic aromatic compound and the pharmaceutically acceptable salt thereof as active components to a patient.

The term treatment, treating or treat in the present application refers to reversing, alleviating the above-mentioned disease or one or more symptoms of a patient suffering from the above-mentioned disease, inhibiting the progress of the above-mentioned disease or one or more symptoms of the patient suffering from the above-mentioned disease, or preventing the above-mentioned disease or one or more symptoms of the patient suffering from the above-mentioned disease.

The patient described in the present application includes all members of the animal kingdom, including but not limited to mammals and humans; and the mammal may be mice, rats, cats, monkeys, dogs, horses, pigs, etc. In a possible implementation, the patient of the present application is a human being.

When the compound represented by Formula 1 of the present application and the pharmaceutically acceptable salt are used for the prevention and/or treatment of tumors, the dosage thereof may be changed according to the route of administration, the age, body weight and condition of the patient, or the type and severity of the disease to be treated.

It can be expected from pharmacodynamic experiments that the drug, which takes the nitrogen-containing polycyclic aromatic compound of the present application and its pharmaceutically acceptable salt as active ingredients, has good medicinal effects on cervical cancer, breast cancer, melanoma, lung cancer, bone marrow cancer and other malignant tumors.

The present application at least has the following beneficial effects:
the nitrogen-containing polycyclic compound provided by the present application is a small molecular compound with a novel structure, which shows excellent broad-spectrum anti-tumor activity, especially, it shows an inhibitory rate comparable to cisplatin for cervical cancer cells HELA, breast cancer cells MCF-7 and MDA-MB-231, melanoma cells A375, lung cancer cells A549 and bone marrow cancer cells SP2/0. And it can be seen from the toxicity test on normal cells 293T and the acute toxicity test on mice that the toxicity of the small molecular compound with the structure of Formula I is much lower than that of cisplatin. Therefore, the compound represented by Formula I may be used as a lead compound for the research of new low-toxicity and high-efficiency broad-spectrum anti-tumor drugs.

### DESCRIPTION OF THE EMBODIMENTS

The present application will be further described in detail below in conjunction with specific embodiments. It should be understood that the following embodiments are only for illustrating and explaining the present application, and should not be construed as limitations on the protection scope of the present application. All technologies implemented based on the above contents of the present application are covered within the scope of protection intended by the present application.

### Synthesis of Compound 1

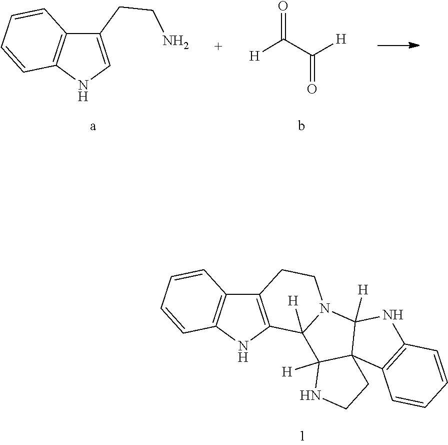

The synthesis steps of the compound 1 include: dissolving tryptamine (compound a) (1 mmol) in dichloromethane, and adding glyoxal (compound b) (0.55 mmol, 0.55 equiv.), 4 Å molecular sieve and trifluoroacetic acid (5% to 10% mol); carrying out the reaction at −5° C. to 5° C. until it is detected by TLC (Thin-Layer Chromatography) that the tryptamine disappears substantially, showing the reaction being over; removing the 4 Å molecular sieve by filtration; concentrating the filtrate; and subjecting the concentrated filtrate to column chromatography separation (dichloromethane:methanol=(30 to 50):1), thereby obtaining a compound 1 (102 mg, yield 60%).

The characterization of compound 1 is as follows:

¹H NMR (600 MHz, CDCl3) δ 10.08 (s, 1H), 7.22 (t, J=8.0 Hz, 3H), 7.07 (d, J=7.4 Hz, 1H), 7.03 (dd, J=14.8, 7.4 Hz, 3H), 6.92 (t, J=7.4 Hz, 1H), 6.73 (t, J=7.4 Hz, 1H), 6.53 (d, J=7.8 Hz, 1H), 5.03 (s, 1H), 4.19 (s, 1H), 4.12 (d, J=3.7 Hz, 1H), 3.22 (d, J=4.8 Hz, 1H), 3.09 (dd, J=18.1, 7.4 Hz, 1H), 3.02 — 2.96 (m, 1H), 2.90 (d, J=7.3 Hz, 2H), 2.70 (d, J=10.8 Hz, 1H), 2.48 (s, 1H), 2.16 (dt, J=13.7, 6.7 Hz, 1H);

¹³C NMR (151 MHz, CDCl3) δ 150.89, 136.82, 129.77, 129.14, 127.04, 123.44, 121.94, 119.72, 119.53, 118.13, 111.88, 111.29, 109.65, 87.86, 71.42, 63.56, 57.32, 48.17, 43.50, 38.92, 21.71;

HRMS-ESI m/z calcd for C₂₂H₂₂N₄ [M+H]⁺ 343.1923, found 343.1920.

Compounds 2 to 22 are prepared with reference to the synthesis steps of compound 1.

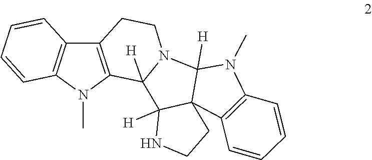

The characterization of compound 2 is as follows:

¹H NMR (600 MHz, CDCl₃) δ 7.46 (d, J=8.1 Hz, 1 H), 7.23 (m, 2 H), 7.19 (m, 2H), 7.10-7.04 (m, 1H), 6.80 (t, J=7.3 Hz, 1H), 6.49 (d, J=7.9 Hz, 1H), 4.72 (s, 1H), 4.31 (d, J=6.8 Hz, 1H), 3.70 (m, 3H), 3.54 (m, 1H), 3.24 (s, 1H), 3.21-3.15 (m, 1H), 3.06-3.01 (m, 1H), 2.96 (s, 3H), 2.83 (m, 1H), 2.62-2.55 (m, 1H), 2.23 (m, 1H), 1.76-1.67 (m, 1H), 1.62-1.56 (m, 1H), 1.48-1.41 (m, 1H);

¹³C NMR (151 MHz, CDCl₃) δ 147.01, 137.64, 137.55, 132.32, 127.13, 126.66, 122.55, 120.49, 118.87, 118.22, 118.02, 109.47, 108.97, 106.76, 96.29, 65.58, 62.00, 58.86, 48.54, 44.94, 37.10, 36.64, 29.37, 22.70;

HRMS-ESI m/z calcd for C₂₄H₂₆N₄ [M+H]⁺ 371.2236, found 371.2230.

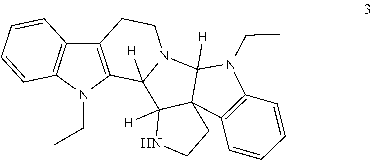

The characterization of compound 3 is as follows:

¹H NMR (600 MHz, CDCl₃) δ 7.49 (d, J=8.4 Hz, 1H), 7.29 (d, J=8.0 Hz, 1H), 7.24 (d, J=7.2 Hz, 1H), 7.20-7.15 (m, 2H), 7.10-7.07 (m, 1H), 6.77 (t, J=7.3 Hz, 1H), 6.49 (d, J=7.8 Hz, 1H), 4.89 (s, 1H), 4.31 (m, 1H), 3.53-3.49 (m, 1H), 3.40-3.36 (m, 1H), 3.33-3.27 (m, 2H), 3.03 (m, 2H), 2.84 (m, 1H), 2.64-2.58 (m, 1H), 2.31 (s, 2H), 2.20-2.15 (m, 2H), 1.64-1.58 (m, 2H), 1.30 (d, J=7.1 Hz, 3H), 1.20 (dd, J=7.1, 3.5 Hz, 3H);

¹³C NMR (151 MHz, CDCl₃) δ 136.28, 132.60, 132.46, 128.25, 122.56, 118.97, 118.24, 116.82, 109.37, 106.30, 97.13, 68.31, 65.73, 44.87, 43.14, 39.15, 29.67, 22.87, 15.50, 12.42;

HRMS-ESI m/z calcd for C₂₆H₃₀N₄ [M+H]⁺ 399.2549, found 399.2547.

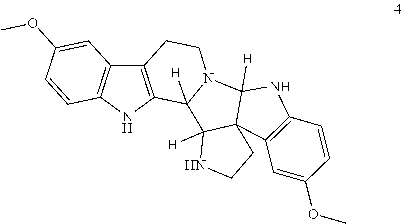

The characterization of compound 4 is as follows:

¹H NMR (400 MHz, CDCl₃) δ 10.00 (s, 1H), 7.19 (d, J=8.7 Hz, 1H), 6.89 (m, 1H), 6.78 (m, 2H), 6.72 (m, 1H), 6.58 (d, J=8.5 Hz, 1H), 5.14 (s, 1H), 4.31 (s, 2H), 3.83 (s, 3H), 3.77 (s, 3H), 3.28 (m, 1H), 3.18 (m, 1H), 3.00 (m, 3H), 2.77 (m, 1H), 2.64 (m, 1H), 2.33 (m, 1H);

¹³C NMR (150 MHz, CDCl₃) δ 154.19, 151.50, 144.79, 135.69, 135.36, 131.07, 128.99, 114.41, 112.82, 112.79, 112.21, 109.63, 109.61, 100.48, 88.43, 70.14, 63.83, 57.23, 55.89, 48.73, 41.05, 37.34, 22.05;

HRMS-ESI m/z calcd for C₂₄H₂₆N₄₂ [M+H]⁺ 403.2134, found 403.2129.

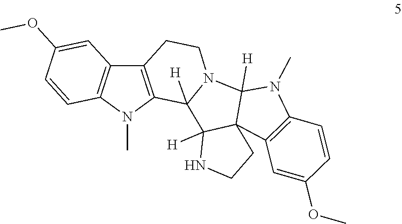

The characterization of compound 5 is as follows:

¹H NMR (600 MHz, CDCl₃) δ 7.15 (d, J=8.8 Hz, 1H), 6.93 (d, J=2.3 Hz, 1H), 6.84 (m, 2H), 6.73 (dd, J=8.4, 2.5 Hz, 1H), 6.42 (d, J=8.5 Hz, 1H), 4.60 (s, 1H), 3.85 (s, 3H), 3.79 (s, 3H), 3.67 (s, 3H), 3.50 (m, 1H), 3.26 (m, 1H), 3.13 (m, 1H), 3.02 (m, 1H), 2.95 (m, 1H), 2.93 (s, 3H), 2.77 (m, 1H), 2.53 (m, 1H), 2.11 (m, 1H), 1.45 (m, 2H);

¹³C NMR (151 MHz, CDCl₃) δ 153.88, 153.39, 147.72, 137.89, 132.98, 129.92, 126.79, 116.27, 113.71, 111.38, 110.44, 109.84, 107.91, 100.35, 87.70, 72.43, 62.93, 58.56, 56.03, 49.03, 44.55, 37.93, 22.96;

HRMS-ESI m/z calcd for C₂₆H₃₀N₄O₂ [M+H]⁺ 431.2447, found 431.2440.

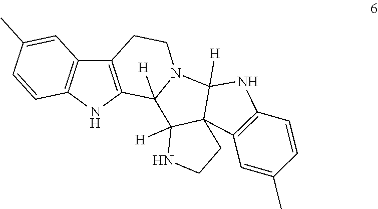

The characterization of compound 6 is as follows:

¹H NMR (600 MHz, CDCl₃) δ 9.42 (s, 1H), 7.22 (s, 1H), 7.14 (d, J=8.2 Hz, 1H), 6.95 (s, 1H), 6.90 (t, J=8.1 Hz, 2H), 6.52 (d, J=7.9 Hz, 1H), 5.00 (s, 1H), 4.24 (d, J=3.7 Hz, 1H), 4.01 (d, J=4.5 Hz, 1H), 3.26 (m, 1H), 3.06-2.90 (m, 4H), 2.73 (m, 1H), 2.42 (s, 3H), 2.38 (m, 1H), 2.29 (s, 3H), 2.12-2.07 (m, 1H);

¹³C NMR (151 MHz, CDCl₃) δ 148.54, 135.20, 130.20, 129.52, 129.25, 128.71, 127.39, 124.07, 123.52, 117.93, 111.56, 110.91, 109.70, 88.13, 71.60, 63.77, 57.61, 48.47, 43.63, 39.18, 29.84, 21.70, 21.61, 20.95;

HRMS-ESI m/z calcd for C₂₄H₂₆N₄ [M+H]⁺ 371.2236, found 371.2230.

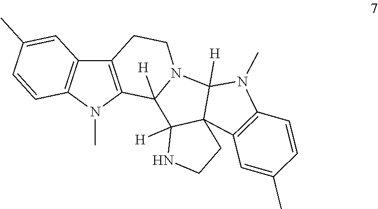

The characterization of compound 7 is as follows:

¹H NMR (600 MHz, CDCl₃) δ 7.14 (d, J=8.8 Hz, 1H), 6.91 (d, J=2.1 Hz, 2H), 6.86 (m, 1H), 6.78 (m, 1H), 6.45 (d, J=8.5 Hz, 1H), 4.67 (s, 1H), 4.34 (s, 1H), 3.84 (s, 3H), 3.80 (s, 3H), 3.69 (s, 3H), 3.51 (d, 1H), 3.02 (m, 1H), 2.92 (s, 3H), 2.79 (m, 1H), 2.31 (m, 2H);

¹³C NMR (151 MHz, CDCl₃) δ 153.27, 135.67, 130.05, 128.86, 128.70, 128.09, 126.83, 126.63, 123.40, 118.59, 118.38, 109.36, 109.16, 100.15, 91.68, 66.86, 63.93, 58.33, 43.97, 43.65, 43.08, 39.78, 38.25, 32.60, 21.61, 20.88;

HRMS-ESI m/z calcd for C₂₆H₃₀N₄ [M+H]⁺ 399.2549, found 399.2541.

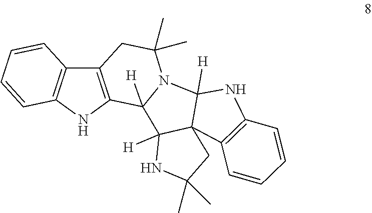

The characterization of compound 8 is as follows:

¹H NMR (600 MHz, CDCl₃) δ 7.42 (m, 1H), 7.25 (d, J=7.4 Hz, 1H), 7.17 (s, 1H), 7.10 (t, J=7.6 Hz, 1H), 7.02 (m, 2H), 6.80 (t, J=7.4 Hz, 1H), 6.61 (d, J=7.8 Hz, 1H), 4.51 (s, 1H), 4.32 (d, J=3.6 Hz, 1H), 3.97 (m, 1H), 2.82 (d, J=14.7 Hz, 1H), 2.64 (d, J=14.7 Hz, 1H), 2.50 (d, J=14.0 Hz, 1H), 2.04 (d, J=14.0 Hz, 1H), 1.54 (s, 3H), 1.53 (s, 3H), 1.26 (s, 3H) 1.25 (s, 3H);

¹³C NMR (151 MHz, CDCl₃) δ 151.04, 137.31, 130.16, 128.88, 127.16, 123.86, 121.87, 119.35, 119.17, 117.85, 111.91, 111.35, 109.38, 87.10, 72.75, 64.50, 64.34, 55.90 , 52.58, 38.22, 29.83, 29.21 , 28.42, 27.29, 22.08;

HRMS-ESI m/z calcd for C₂₆H₃₀N₄ [M+H]⁺ 399.2549, found 399.2543.

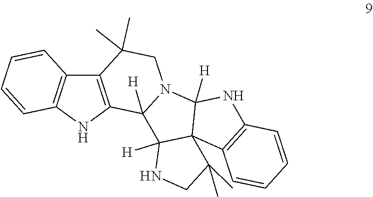

The characterization of compound 9 is as follows:

¹H NMR (600 MHz, CDCl₃) δ 7.64 (d, J=7.9 Hz, 1H), 7.32 (d,J =7.8 Hz, 1H), 7.29 (d, J =7.8 Hz, 1H), 7.12 (m, 2H), 7.05 (t, J=7.2 Hz, 1H), 6.76 (t, J=7.4 Hz, 1H), 6.63 (d, J=7.8 Hz, 1H), 5.21 (s, 1H), 4.44 (s, 1H), 4.10 (d, J=2.8 Hz, 1H), 3.06 (m, 3H), 2.84 (m, 2H), 1.47 (s, 3H×2), 1.26 (s, 3H×2);

¹³C NMR (151 MHz, CDCl₃) δ 151.56, 137.54, 129.82, 128.51, 126.91, 126.02, 124.01, 122.25, 120.14, 119.46), 119.28, 118.51, 112.59, 109.91, 80.52, 71.00, 69.09, 57.73, 51.73, 51.19, 50.96, 40.89, 34.86, 30.34, 24.70, 23.40;

MS-ESI m/z calcd for C₂₆H₃₀N₄ [M+Na]⁺ 399.2549, found 399.2543.

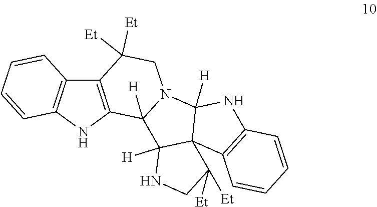

The characterization of compound 10 is as follows:

¹H NMR (600 MHz, CDCl₃) δ 10.07 (s, 1H), 7.57 (d, J=8.0 Hz, 1H), 7.36 (dd, J=7.7, 4.3 Hz, 2H), 7.13 (dd, J=14.2, 7.0 Hz, 2H), 7.03 (t, J=7.5 Hz, 1H), 6.78 (t, J=7.4 Hz, 1H), 6.64 (d, J=7.7 Hz, 1H), 4.83 (s, 1H), 4.09 (s, 1H), 2.97 (d, J=11.6 Hz, 1H), 2.85 (d, J=11.6 Hz, 2H), 2.28 (m, 1H), 2.03 (m, 1H), 1.94 (m, 2H), 1.85 (m, 2H), 1.73 (m, 3H), 1.50 (m, 1H), 1.00 (t, J=7.1 Hz, 3H), 0.91 (t, J=7.5 Hz, 3H), 0.69 (t, J=7.4 Hz, 3H), 0.56 (t, J=7.5 Hz, 3H);

¹³C NMR (151 MHz, CDCl₃) δ 151.56, 137.54, 129.82, 128.51, 126.91, 126.02, 124.01, 122.25, 120.14, 119.46, 119.28, 118.51, 112.59, 109.91, 80.52, 71.00, 69.09, 57.73, 51.19, 50.96, 40.89, 34.86, 30.34, 24.70, 23.40, 10.42, 9.03, 8.24, 7.78;

HRMS-ESI m/z calcd for C₃₀H₃₈N₄ [M+H]⁺ 455.3175, found 455.3169.

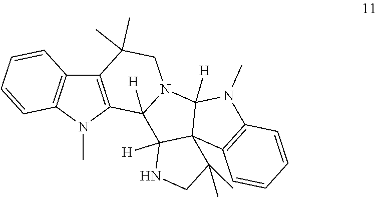

The characterization of compound 11 is as follows:

¹H NMR (600 MHz, CDCl₃) δ 7.68 (d, J=8.0 Hz, 1H), 7.32 (d, J=7.4 Hz, 1H), 7.25 (s, 1H), 7.19 (m, 3H), 7.07 (t, J=7.4 Hz, 1H), 6.73 (t, J=7.4 Hz, 1H), 6.49 (d, J=7.9 Hz, 1H), 4.76 (s, 1H), 4.32 (d, J=2.0 Hz, 1H), 4.11 (d, J=2.9 Hz, 1H), 3.69 (s, 3H), 3.10 (d, J=9.8 Hz, 1H), 3.06-3.01 (m, 3H), 3.00 (s, 3H), 2.83 (d, J=11.0 Hz, 1H), 1.52 (s, 3H×2), 1.47 (s, 3H×2);

¹³C NMR (151 MHz, CDCl₃) δ 154.35, 137.76, 129.29, 126.48, 125.20, 121.55, 120.36, 120.03, 119.02, 117.45, 109.56, 107.01, 89.30, 70.10, 60.96, 59.35, 58.91, 44.40, 36.01, 34.23, 30.89, 29.59), 27.03, 26.02, 22.31;

HRMS-ESI m/z calcd for C₂₈H₃₄N₄ [M+H]⁺ 427.2862, found 427.2859.

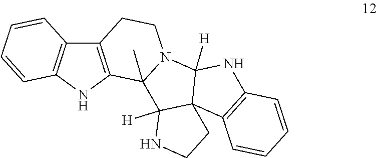

The characterization of compound 12 is as follows:

¹H NMR (600 MHz, CDCl₃) δ 9.31 (s, 1H), 7.46 (d, J=7.8 Hz, 1H), 7.35 (d, J=8.0 Hz, 1H), 7.14-7.05 (m, 4H), 6.78 (t, J=7.5 Hz, 1H), 6.62 (d, J=7.8 Hz, 1H), 5.01 (s, 1H), 3.48 (s, 1H), 3.34-3.28 (m, 2H), 3.12 (m, 1H), 3.01-2.94 (m, 1H), 2.69 (d, J=7.8 Hz, 1H), 2.64 (m, 1H), 2.14 (m, 1H), 2.04 (m, 1H), 1.52 (s, 3H), 1.28 (s, 3H);

¹³C NMR (151 MHz, CDCl₃) δ150.36, 136.73, 128.65, 127.05, 125.10, 121.81, 119.26, 118.94, 118.18, 111.67, 110.35, 85.89, 65.75, 65.60, 45.16, 40.51, 39.33, 29.69, 22.38, 19.52;

HRMS-ESI m/z calcd for C₂₄H₂₆N₄ [M+H]⁺ 371.2236, found 371.2230.

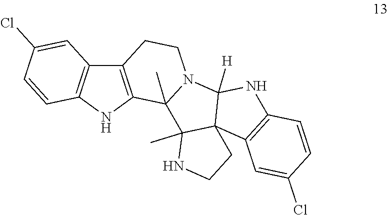

The characterization of compound 13 is as follows:

¹H NMR (600 MHz, CDCl₃) δ 7.43 (d, J=1.7 Hz, 1H), 7.25 (s, 1H), 7.09 (dd, J=8.6, 1.8 Hz, 1H), 7.04 (dd, J=8.3, 2.0 Hz, 1H), 7.01 (d, J=1.8 Hz, 1H), 6.57 (d, J=8.3 Hz, 1H), 4.88 (s, 1H), 3.31 (m, 2H), 3.16 (m, 1H), 3.00 (m, 1H), 2.60 (m, 2H), 2.00 (m, 1H), 1.85 (m, 1H), 1.39 (s, 3H), 1.35 (s, 3H);

¹³C NMR (151 MHz, CDCl₃) δ 148.90, 134.94, 134.11, 128.30, 128.20, 125.15, 125.00, 123.67, 121.89, 117.76, 112.42, 111.39, 107.87, 85.16, 65.84, 65.49, 63.85, 50.99, 46.42, 42.07, 39.55, 29.85, 23.94, 22.16, 17.91;

HRMS-ESI m/z calcd for C₂₄H₂₄Cl₂N₄ [M+H]⁺ 439.1456, found 439.1450.

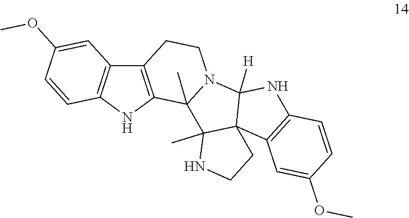

The characterization of compound 14 is as follows:

¹H NMR (600 MHz, CDCl₃) δ 9.21 (s, 1H), 7.27 (d, J=7.2 Hz, 1H), 6.98 (s, 1H), 6.83 (m, 1H), 6.70 (m, 2H), 6.65 (m, 1H), 4.84 (s, 1H), 3.89 (s, 3H), 3.79 (s, 3H), 3.41 (dd, J=13.9, 6.5 Hz, 1H), 3.33 (m, 1H), 3.15 (m, 1H), 3.7 (m, 1H), 2.65 (dd, J=15.6, 5.2 Hz, 1H), 2.51 (m, 1H), 1.96 (m, 1H), 1.81 (m, 1H), 1.40 (s, 3H), 1.35 (s, 3H);

¹³C NMR (600 MHz, CDCl₃) δ 153.93, 144.31, 140.63, 135.15, 131.60, 127.62, 112.74, 112.22, 111.93, 111.68, 111.25, 107.45, 100.49, 84.69 76.28, 66.00, 65.15, 56.14, 46.85, 42.35, 39.76, 24.71, 23.14, 17.37;

HRMS-ESI m/z calcd for C₂₆H₃₀N₄O₂ [M+H]⁺ 431.2447, found 431.2476.

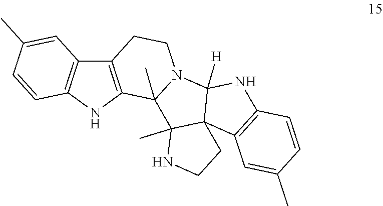

The characterization of compound 15 is as follows:

¹H NMR (600 MHz, CD₃OD) δ7.25 (m, 2H), 7.08 (s, 1H), 6.94 (m, 2H), 6.49 (d, J=7.9 Hz, 1H), 5.16 (s, 1H), 3.37-3.25 (m, 6H), 3.05-2.98 (m, 1H), 2.72 (dd, J=14.8, 3.6 Hz, 1H), 2.58-2.49 (m, 1H), 2.40 (s, 3H), 2.28 (s, 3H), 1.73 (s, 3H), 1.22 (s, 3H);

¹³C NMR (151 MHz, CD₃OD) δ 150.72, 136.86, 136.24, 130.85, 129.38, 129.11, 128.54, 128.51, 126.57, 124.59, 118.88, 112.16, 111.79, 110.95, 88.83, 79.19, 67.48, 67.03, 45.01, 40.07, 39.22, 23.21, 21.59, 20.96, 19.79, 17.40;

HRMS-ESI m/z calcd for C₂₆H₃₀N₄ [M+H]⁺ 399.2549, found 399.2544.

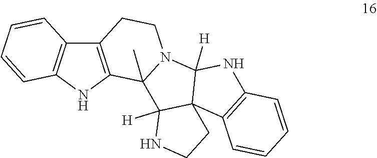

The characterization of compound 16 is as follows:

¹H NMR (600 MHz, CDCl₃) δ 9.44 (s, 1H), 7.50 (d, J=7.8 Hz, 1H), 7.38 (d, J=8.0 Hz, 1H), 7.16 (t, J=7.2 Hz, 1H), 7.09 (m, 3H), 6.81 (t, J=7.3 Hz, 1H), 6.72 (d, J=7.6 Hz, 1H), 4.58 (s, 1H), 4.53 (s, 1H), 3.92 (s, 1H), 3.38 (dd, J=14.4, 6.1 Hz, 1H), 3.24 (m, 1H), 3.10-3.03 (m, 2H), 2.64 (dd, J=15.9, 5.3 Hz, 1H), 2.12-2.05 (m, 1H), 1.92-1.87 (m, 1H), 1.66 (m, 1H), 1.51 (s, 3H);

¹³C NMR (151 MHz, CDCl₃) δ 149.01, 137.68, 136.93, 136.76, 127.90, 127.28, 122.38, 121.51, 120.04, 119.13, 118.16, 111.34, 107.74, 82.43, 80.14, 62.57, 61.81, 48.45, 41.53, 39.40, 29.73, 16.11;

HRMS-ESI m/z calcd for C₂₃H₂₄N₄ [M+H]⁺ 357.2079, found 357.2075.

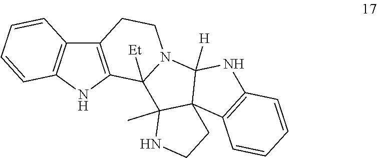

The characterization of compound 17 is as follows:

¹H NMR (600 MHz, CDCl₃+CD₃OD (˜5/1)) δ 7.43 (d, J=7.8 Hz, 1H), 7.38 (d, J=8.1 Hz, 1H), 7.10 (m, 3H), 7.03 (m, 1H), 6.72 (t, J=7.5 Hz, 1H), 6.56 (d, J=7.9 Hz, 1H), 5.13 (s, 1H), 3.45 (m, 1H), 3.12 (m, 1H), 2.93 (m, 1H), 2.81 (m, 1H), 2.54 (m, 1H), 2.28 (m, 1H), 1.72 (s, 2H), 1.60 (m, 2H), 1.19 (s, 3H), 0.68 (t, J=7.3 Hz, 3H);

¹³C NMR (151 MHz, CDCl₃+CD₃OD (˜5/1)) δ 150.83, 137.22, 129.62, 126.67, 126.39, 125.13, 122.70, 119.53, 118.78, 118.19, 112.09, 109.96, 87.44, 79.47, 65.80, 63.57, 44.20, 37.74, 30.25, 29.72, 21.93, 17.74, 11.14;

HRMS-ESI m/z calcd for C₂₅H₂₈N₄ [M+H]⁺ 385.2392, found 385.2387.

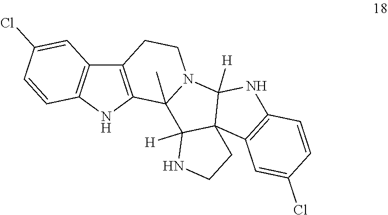

The characterization of compound 18 is as follows:

¹H NMR (600 MHz, CDCl₃) ≢7 9.55 (s, 1H), 7.43 (d, J=1.7 Hz, 1H), 7.25 (s, 1H), 7.09 (dd, J=8.6, 1.8 Hz, 1H), 7.04 (dd, J=8.3, 2.0 Hz, 1H), 7.01 (d, J=1.8 Hz, 1H), 6.57 (d, J=8.3 Hz, 1H), 4.88 (s, 1H), 3.31 (dt, J=15.8, 5.7 Hz, 2H), 3.16 (dt, J=11.6, 7.2 Hz, 1H), 3.00 (ddd, J=16.1, 10.8, 7.6 Hz, 1H), 2.64 (dd, J=15.7, 3.9 Hz, 1H), 2.60-2.56 (m, 1H), 2.05-1.96 (m, 2H), 1.85 (dt, J=13.5, 6.9 Hz, 1H), 1.39 (s, 3H), 1.35 (s, 3H);

¹³C NMR (151 MHz, CDCl₃) ≢7 148.90, 134.94, 134.11, 128.30, 128.20, 125.15, 125.00, 123.67, 121.89, 117.76, 112.42, 111.39, 107.87, 85.16, 65.84, 65.49, 50.99, 46.42, 42.07, 39.55, 23.94, 17.91;

HRMS-ESI m/z calcd for C₂₃H₂₂Cl₂N₄ [M+H]⁺ 425.1300, found 425.1289.

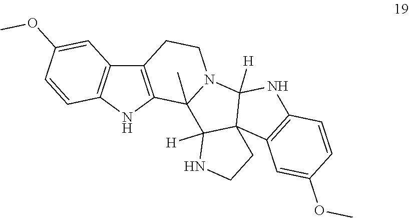

The characterization of compound 19 is as follows:

¹H NMR (600 MHz, CDCl₃) δ 7.28 (d, J=8.7 Hz, 1H), 6.94 (m, 2H), 6.80 (dd, J=8.7, 2.4 Hz, 1H), 6.68 (m, 2H), 4.71 (s, 1H), 4.08 (s, 1H), 3.85 (s, 3H), 3.77 (s, 3H), 3.37 (dd, J=13.9, 6.2 Hz, 1H), 3.14 (m, 2H), 3.03 (m, 1H), 2.62 (dd, J=15.6, 4.9 Hz, 1H), 2.37 (m, 1H), 2.00 (m, 2H), 1.52 (s, 3H);

¹³C NMR (151 MHz, CDCl₃) δ 154.95, 154.12, 142.94, 136.72, 131.99, 127.49, 113.93, 112.45, 112.40, 111.79, 109.54, 108.84, 100.54, 84.45, 77.77, 63.02, 62.80, 56.19, 56.12, 47.63, 39.83, 39.29, 28.20, 17.47;

HRMS-ESI m/z calcd for C₂₅H₂₈N₄O₂ [M+H]⁺ 417.2291, found 417.2287.

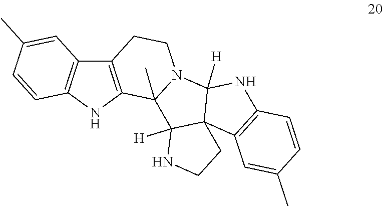

The characterization of compound 20 is as follows:

¹H NMR (600 MHz, CDCl₃) δ 7.25 (m, 2H), 7.00 (s, 1H), 6.96 (d, J=8.7 Hz, 1H), 6.90 (d, J=7.8 Hz, 1H), 6.60 (d, J=7.9 Hz, 1H), 4.70 (s, 1H), 4.02 (s, 1H), 3.33 (m, 1H), 3.20 (m, 1H), 3.12 (m, 1H), 3.00 (m, 1H), 2.62 (dd, J=15.7, 4.9 Hz, 1H), 2.43 (s, 3H), 2.35 (m, 1H), 2.27 (s, 3H), 1.99 (m, 1H), 1.93 (m, 1H), 1.47 (s, 3H);

¹³C NMR (151 MHz, CDCl₃) δ 146.95, 136.60, 135.12, 129.62, 128.90, 128.57, 127.40, 123.58, 123.34, 117.98, 111.35, 111.18, 108.39, 84.23, 78.27, 62.73, 47.82, 40.43, 39.36, 27.86, 21.59, 21.05, 17.66;

HRMS-ESI m/z calcd for C₂₅H₂₈N₄ [M+H]⁺ 385.2392, found 385.2387.

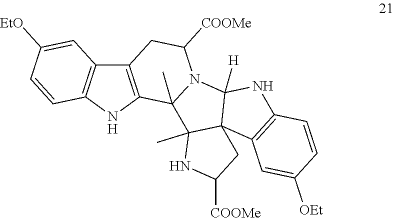

The characterization of compound 21 is as follows:

¹H NMR (600 MHz, CDCl₃) δ 9.75 (s, 1H), 7.24 (s, 1H), 6.91 (d, J=2.0 Hz, 1H), 6.79 (dd, J=8.7, 2.3 Hz, 1H), 6.65 (dd, J=8.5, .3 Hz, 1H), 6.59 (d, J=2.2 Hz, 1H), 6.5 (d, J=8.5 Hz, 1H), 5.15 (s, 1H), 4.49 (dd, J=11.6, 7.2 Hz, 1H), 4.15 (dd, J=11.8, 5.0 Hz, 1H), 4.06 (m, 2H), 3.94 (m, 2H), 3.83 (s, 3H), 3.47 (s, 3H), 3.31 (dd, J=15.7, 11.9 Hz, 1H), 2.87 (dd, J=15.8, 5.0 Hz, 1H), 2.22 (dd, J=12.9, 7.1Hz, 1H), 1.42 (t, J=7.0 Hz, 3H), 1.37 (t, J=7.0 Hz, 3H), 1.25 (s, 3H), 1.21 (s, 3H);

¹³C NMR (151 MHz, CDCl₃) δ 174.25, 173.69, 153.13, 152.32, 144.54, 139.94, 132.60, 131.65, 127.06, 113.84, 112.29, 112.26, 111.98, 110.00, 105.42, 101.20, 81.72, 75.28, 67.98, 65.31, 64.50, 64.21, 61.75, 52.25, 52.05, 42.99, 29.67, 24.26, 22.05, 20.26, 15.11, 15.02;

HRMS-ESI m/z calcd for C₃₂H₃₉N₄O₆ [M+H]⁺ 575.2870, found 574.2791.

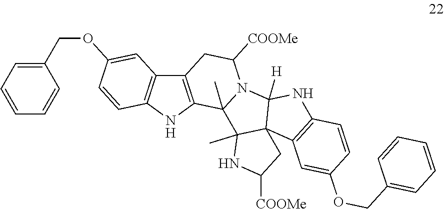

The characterization of compound 22 is as follows:

¹H NMR (600 MHz, CDCl₃) δ 9.72 (s, 1H), 7.40 (d, J=7.4 Hz, 2H), 7.31 (m, 6H), 7.24 (t, J=6.7 Hz, 2H), 7.18 (m, 2H), 6.93 (s, 1H), 6.80 (dd, J=8.7, 2.0 Hz, 1H), 6.67 (dd, J=8.5, 2.1 Hz, 1H), 6.54 (d, J=1.8 Hz, 1H), 6.43 (d, J=8.5 Hz, 1H), 5.07 (s, 1H), 5.02 (s, 2H), 4.90 (q, J=11.5 Hz, 2H), 4.36 (dd, J=11.6, 7.2 Hz, 1H), 4.08 (dd, J=11.8, 4.9 Hz, 1H), 3.76 (s, 3H), 3.40 (s, 3H), 3.24 (m, 1H), 2.79 (m, 1H), 2.12 (m, 1H), 1.37 (s, 4H), 1.30 (m, 1H), 1.10 (s, 3H);

¹³C NMR (151 MHz, CDCl₃) δ 173.18, 153.65, 137.97, 137.24, 133.00, 129.88, 128.82, 128.72, 128.60, 128.12, 127.81, 127.76, 127.62, 127.48, 127.14, 126.51, 113.75, 113.33, 112.82, 102.07, 83.27, 71.46, 71.15, 52.61, 39.07, 33.43, 32.07, 26.92, 26.12, 22.82, 14.22, 11.04;

HRMS-ESI m/z calcd for C₄₂H₄₃N₄O₆ [M+H]⁺ 699.3183, found: 699.3190.

### Biological Activity Evaluation

### 1) Anti-Tumor Activity Experiment

### 1. Determination of the Inhibitory Rates of Drugs on Cells A375, A549, MDA-MB-231 and SP2/0 by a CCK-8 Method

a. Cells and Drugs
Cell lines: human melanoma A375 (provided by Institute of Pharmacology, China Academy of Sciences), human lung cancer cell A549, human triple negative breast cancer MDA-MB-231, and mouse myeloma SP2/0;
Experimental drug: compound 14 synthesized by the aforementioned method; and
Positive control drug: cisplatin.
   b. Reagents
CCK8 reagent kit, purchased from TargetMol China; Annexin V-FITC/PI double staining cell apoptosis detection kit, purchased from Seven Biotech Company.
   c. Instruments
Multi-function microplate reader FLUOstar Omega, purchased from BMG LABTECH, Germany;
Flow cytometer, purchased from BD Company, United States.
   d. Experimental Method

The cells in logarithmic phase with good condition are taken and digested with trypsin, and the prepared culture medium is added to prepare a cell suspension, the cell density being adjusted to 5×10⁴ cells/mL. The digested cells are inoculated into a 96-well plate, in which 100 μL of cell suspension is added to each well, and incubated overnight in a cell incubator at 37° C. with 5% CO₂. After the cells grow adhered to the wall, the blank control group and the compound groups to be tested with different concentration gradients are set up respectively. Each group is provided with five parallel replicates, and 150 μL of drug is added to each well, followed by cultivation for further 48 hours. 10 μL of CCK8 solution is added to each well, shaked and mixed, and then the mixture is put into an incubator for cultivation for further 1.5 h. The optical density value (OD) is measured by using a multifunctional enzyme-linked immunosorbent assay instrument, the wavelength setting to 450 nm. The experiment is repeated for three times, the cell survival rates of different drug concentrations in each group are calculated and counted according to a formula: cell survival rate=(experimental group−blank control group/control group)×100%, and the results are calculated with GraphPad Prism software.

The drug is applied by respectively preparing cisplatin and compound 14 into mother liquors, which are sequentially added from low to high concentrations (0.5 μM to 100 μM) to tumor cell lines that are in logarithmic phase and have good conditions, and the cell survival rate is detected by the CCK-8 method. The IC₅₀ values of the inhibition of compound 14 and cisplatin on four types of tumor cells A375, A549, MDA-MB-231 and SP2/0 are shown in Table 1.

TABLE 1

| | IC₅₀ value of | IC₅₀ value of | IC₅₀ value of | IC₅₀ value of |
|---|---|---|---|---|
| | the inhibition | the inhibition | the inhibition | the inhibition |
| | on A375 (μM) | on MDA-MB-231 (μM) | on A549 (μM) | on SP2/0 (μM) |
| Compound 14 | 17.08 ± 1.45 | 26.04 ± 1.43 | 30.80 ± 1.72 | 15.13 ± 2.91 |
| Cisplatin | 16.35 ± 0.36 | 23.37 ± 1.75 | 35.84 ± 1.89 | 17.45 ± 1.24 |

As can be seen from the data in Table 1, the IC50 values of the inhibition of compound 14 on A375, MDA-MB-231, A549, SP2/0, HELA, and MCF-7 cells are comparable to that of cisplatin. It can be seen that compound 14 shows a broad-spectrum antitumor activity comparable to cisplatin.

### 2. Determination of the Cell Inhibitory Rate of Drugs on HELA, MCF-7 and Human Normal Cell 293T by a MTT Method

Two cancer cell lines HELA and MCF-7 in the logarithmic phase and the human normal cell line 293T are taken and the cells thereof are counted with a cell counting plate, and then the concentration of the cell suspension is adjusted to 10⁵ cells/mL. The cells are inoculated into a 96-well plate with 100 μL of cell suspension for each well. After the cell suspension is evenly distributed, and the plate is placed into a CO₂ incubator for incubation. The drug to be tested is added after the cells grow uniformly and have spread on all the bottom of the culture dish. DMEM culture solution is used as a negative control group (the drug to be tested is completely dissolved in the culture solution without dissolving it in DMSO), and cisplatin is used as a positive control group. For each drug to be tested and control groups, 100 μL of the drug to be tested is added to each well; and four parallel replicates are set for each concentration. After 48 h cultivation, 10 μL MTT is added for another 4 h cultivation, and the supernatant is discarded. 100 μL analytically pure dimethyl sulfoxide is added, and shaken evenly on a shaking table. After the solution is completely uniform, its absorbance is measured at a wavelength of 490 nm with a microplate reader. The experiment is repeated twice.

In the above, the drugs to be tested used in the determination of human normal cell line 293T are compounds 1 to 22 of the present application.

The experimental results show that: the IC₅₀ value of the inhibition of cisplatin on normal cell 293T is 17.29 μM, while the IC₅₀ values of the inhibition of compound 1 to compound 22 of the present application on the human normal cell 293T are all greater than 40 μM, in which the IC₅₀ value of the inhibition of compound 15 on the human normal cell 293T can reach up to 133.63 μM. It can be seen that the cytotoxicity of the compounds of the present application to human normal cell 293T is far less than that of cisplatin.

The drugs to be tested which are used to determine the inhibitory rate on cancer cell HELA are compound 15 and compound 20 of the present application, and the drug to be tested which is used to determine the inhibitory rate on cancer cell MCF-7 is compound 15 of the present application. The IC₅₀ values of the inhibition of the above-mentioned drugs and cisplatin on the two types of tumor cells HELA and MCF-7 are shown in Table 2.

TABLE 2

| | IC₅₀ value of | IC₅₀ value of |
|---|---|---|
| | the inhibition | the inhibition |
| | on HELA (μM) | on MCF-7 (μM) |
| Compound 15 | 23.56 | 29.78 |
| Compound 20 | 40.13 | — |
| Cisplatin | 13.15 | 14.18 |

As can be seen from the data in Table 2, the inhibitory activity of the compounds 15 and 20 of the present application to HELA and MCF-7 is slightly lower than that of cisplatin, but the IC₅₀ values of the inhibition of the two compounds of the present application and cisplatin on HELA and MCF-7 are on the same order of magnitude, and the difference in activity thereof is not significant.

### 2) Flow Cytometric Detection of Cell Apoptosis

The cells that are in logarithmic phase and have good condition are taken, digested with trypsin, the prepared culture medium is added to prepare a cell suspension, and the cell density is adjusted to 5×10⁴ cells/mL. The digested cells are inoculated in 6-well plate, in which 2 mL cell suspension is added to each well, and incubated overnight in a cell incubator at 37° C. with 5% CO₂. After the cells are adhered to the wall, a blank control group and the group of experimental drug to be tested with different concentration gradients are set respectively. 2 mL drug is added to each well for further cultivation for 48 h, and EDTA-free trypsin is used for cell digestion and the digested cells are collected. For the apoptosis detection, the collected cells are required to be adjusted to the cell density of 1×10⁶ cells/mL, put into a 5 mL centrifuge tube and centrifuged at 1000 rpm for 5 min, and then washed with PBS for three times. 100 μL Binding Buffer is added to resuspend the cells, and except for the single-stained control and blank control cells, 5 μL FITC and 5 μL PI are added to each tube, mixed gently, and incubated at room temperature in the dark for 15 min. 400 μL Binding Buffer is added into each well before testing on the machine, and mixed and tested on the machine.
Blank control: drug-induced apoptotic cells—without adding a fluorescent reagent (the voltage of scattered light is adjusted to determine the FSC SSC of the population).
Negative control: normal cells (without drug treatment)—added with all the fluorescent staining reagents of the experiment (at the lower left corner, the voltage of the two fluorescent channels is determined).
Single-stained control: one tube each for FITC and PI, positive control for compensation adjustment: drug-induced apoptotic cells or heat-induced apoptotic cells are added with all fluorescent reagents of the experiment (confirming that there is no problem with reagents, instruments and operations).

The test results are shown in Table 3.

TABLE 3

| | | Concen- | Apoptosis | Apoptosis |
|---|---|---|---|---|
| | | tration | in early | in late |
| Cell line | Name | (μM) | stage (%) | stage (%) |
| A375 | Human | 0 | 2.67 ± 2.25 | 3.24 ± 1.71 |
| | melanoma | 10 | 27.41 ± 1.87 | 10.03 ± 1.87 |
| | | 20 | 34.09 ± 3.2 | 13.17 ± 2.30 |
| MDA-MB-231 | Human triple | 0 | 1.03 ± 0.05 | 5.68 ± 0.03 |
| | negative | 15 | 9.21 ± 1.14 | 13.41 ± 1.24 |
| | breast | 30 | 17.70 ± 3.31 | 16.33 ± 0.72 |
| | cancer | 45 | 20.83 ± 2.79 | 23.66 ± 1.70 |
| A549 | Human lung | 0 | 0.35 ± 0.12 | 0.81 ± 0.15 |
| | cancer cell | 10 | 4.12 ± 1.73 | 9.70 ± 1.18 |
| | | 30 | 16.23 ± 0.90 | 16.08 ± 0.62 |
| | | 60 | 41.28 ± 5.36 | 27.32 ± 1.22 |
| SP2/0 | Mouse | 0 | 0.82 ± 0.02 | 0.27 ± 0.01 |
| | myeloma | 10 | 13.15 ± 0.57 | 13.44 ± 1.50 |
| | | 20 | 35.97 ± 3.37 | 26.65 ± 1.64 |
| | | 30 | 58.56 ± 1.26 | 39.36 ± 0.87 |

As can be seen from the data in Table 3, the compound 14 may induce apoptosis in early and late stages of cells A375, MDA-MB-231, A549, SP2/0, and with the increase of drug concentration, the apoptosis rate of the cells gets higher. In particular, it shows a stronger apoptotic effect on mouse myeloma cells, and the total apoptosis rate of SP2/0 cells at early and late stages reaches up to 97% at a drug concentration of 30 μM.

### 3) Mouse Toxicity Test

12 KM mice with body weight 18 g to 22 g and age of 4 to 6 weeks are taken and fed adaptively for 7 days. All mice may eat freely and be provided with sufficient fresh drinking water. The feeding environment for mice meets the standard of SPF laboratory animal level environmental facility.

The KM mice are randomly divided into 6 groups, and are separately intraperitoneally injected with a solution (solvent DMSO) at an injection volume of 20 mL/kg, where the first group to the fourth group are injected with the solution of the compound 14, the dose being 20, 40, 60, and 80 mg/kg; the fifth group is a positive control group injected with cisplatin solution, the cisplatin dose (calculated by cisplatin as an effective ingredient) being 20 mg/kg; and the sixth group is a negative control group only injected with the solvent. The behavioral changes after administration and death within 14 days of the animals are observed.

It can be found by observation that the animals in the experimental group injected with 20 mg/kg compound 14 have no abnormalities, and the weight gain of the animals is not significantly different from that of the normal control group; in the experimental group injected with 20 mg/kg cisplatin, the weight of mice is significantly reduced three days after administration, the animal activity is reduced, the fur is erect, the spirit is listless, and one animal dies on the sixth day; in the experimental group injected with 40 mg/kg compound 14, the weight gain of the animal is slower compared with the negative control group after administration, and the behavior and spirit of the animals are normal, no death occurs during the observation period; in the experimental group injected with 60 mg/kg compound 14, one animal dies on the sixth day, and in the experimental group injected with 80 mg/kg compound 14, two animals die on the second day after administration. Therefore, it can be inferred that the lethal dose of compound 14 is about 60 mg/kg, and the lethal dose of cisplatin is about 20 mg/kg.

The embodiments of the present application have been described above. However, the present application is not limited to the above-mentioned embodiments. Any modifications, equivalent replacements, improvements, etc. made within the spirit and principles of the present application shall be included within the protection scope of the present application.

What is claimed is:

1. A nitrogen-containing polycyclic aromatic compound and a pharmaceutically acceptable salt thereof, the compound having a structure shown in Formula I: 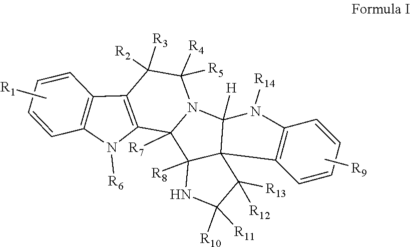
wherein, R₁ and R₉ are each independently selected from a group consisting of: H, C₁₋₆ alkyl, C₁₋₆ alkoxy, benzyloxy, and halogen;
R₂, R₃, R₄, R₅, R₁₀, R₁₁, R₁₂, and R₁₃ are each independently selected from a group consisting of: H, C₁₋₆ alkyl, and —COORₓ; Rₓ is selected from C₁₋₆ alkyl;
R₆ and R₁₄ are each independently selected from H or C₁₋₆ alkyl;
R₇ and R₈ are each independently selected from H or C₁₋₆ alkyl.

2. The nitrogen-containing polycyclic aromatic compound and the pharmaceutically acceptable salt thereof according to claim 1, wherein the C₁₋₆ alkyl is selected from C₁₋₃ alkyl and the C₁₋₆ alkoxy is selected from C₁₋₃ alkoxy.

3. The nitrogen-containing polycyclic aromatic compound and the pharmaceutically acceptable salt thereof according to claim 2, wherein R₁ and R₉ are each independently selected from one of H, methyl, ethyl, ethoxy, benzyloxy, and chlorine; and/or,
R₂, R₃, R₄, R₅, R₁₀, R₁₁, R₁₂, and R₁₃ are each independently selected from one of H, methyl, ethyl, and —COOMe; and/or,
R₆ and R₁₄ are each independently selected from a group consisting of H, methyl and ethyl; and/or,
R₇ and R₈ are each independently selected from a group consisting of H, methyl and ethyl.

4. The nitrogen-containing polycyclic aromatic compound and a pharmaceutically acceptable salt thereof according to claim 3, wherein R₂, R₃, R₄, R₅, R₁₀, R₁₁, R₁₂, and R₁₃ are each selected from H; or,
R₂, R₃, R₁₂, and R₁₃ are each selected from H, and R₄, R₅, R₁₀, and R₁₁ are each selected from methyl or ethyl; or,
R₂, R₃, R₁₂, and R₁₃ are each selected from methyl, and R₄, R₅, R₁₀, R₁₁ are each selected from H; or,
R₂, R₃, R₁₂, and R₁₃ are each selected from H, R₄ and R₅ are each selected from H and —COOMe, and R₁₀ and R₁₁ are each selected from H and —COOMe.

5. The nitrogen-containing polycyclic aromatic compound and the pharmaceutically acceptable salt thereof according to claim 3, wherein R₇ and R₈ are each selected from H or methyl; or,
R₇ is selected from methyl, and R₈ is selected from H; or,
R₇ is selected from ethyl, and R₈ is selected from methyl.

6. The nitrogen-containing polycyclic aromatic compound and the pharmaceutically acceptable salt thereof according to claim 3, wherein R₁ and R₉ are each selected from one of H, methyl, ethyl, ethoxy, benzyloxy and chlorine; and/or,
R₆ and R₁₄ are each selected from a group consisting of H, methyl and ethyl.

7. The nitrogen-containing polycyclic aromatic compound and the pharmaceutically acceptable salt thereof according to claim 1, wherein R₁ and R₉ are each located at a position 5 of its indole ring.

8. A preparation method for the nitrogen-containing polycyclic aromatic compound according to claim 1, comprising the following steps: 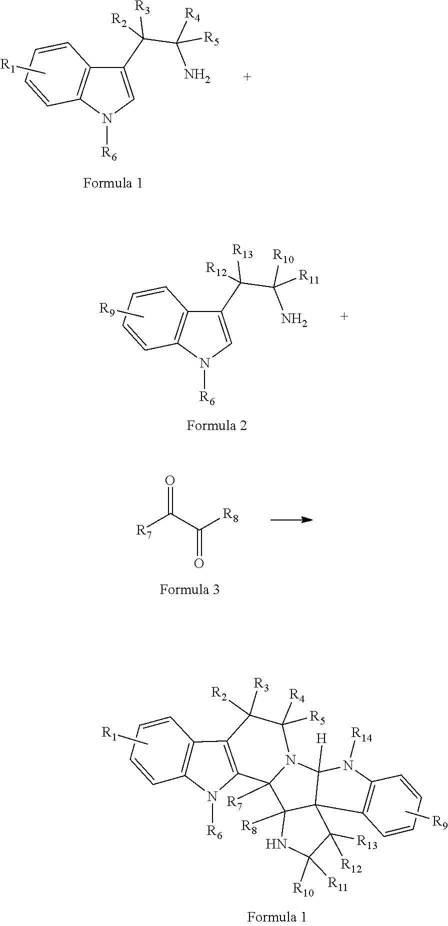
subjecting a compound of Formula 1, a compound of Formula 2 and a compound of Formula 3 to a reaction under an action of acid catalyst to obtain the compound represented by Formula I.

9. The preparation method according to claim 8, wherein a molar ratio of the compound of Formula 1, the compound of Formula 2, and the compound of Formula 3 is 1:1:(1 to 1.4).

10. The preparation method according to claim 8, wherein a temperature of the reaction is −20° C. to 60° C.

11. The preparation method according to claim 8, wherein a system of the reaction further comprises a solvent, and the solvent is at least one selected from a group consisting of a chlorinated solvent, an alcohol solvent, and an ether solvent.

12. The preparation method according to claim 8, wherein the acid catalyst is at least one selected from a group consisting of glacial acetic acid, trifluoroacetic acid, hydrochloric acid, and p-toluenesulfonic acid.

13. A pharmaceutical composition comprising the nitrogen-containing polycyclic aromatic compound according to claim 1 and pharmaceutically acceptable pharmaceutical excipients.

14. The pharmaceutical composition according to claim 13, wherein a dosage form of the pharmaceutical composition comprises at least one of oral formulations, injection formulations or suppositories.

15. Use of the nitrogen-containing polycyclic aromatic compound according to claim 1 in the preparation of an anti-tumor drug.

16. The use according to claim 15, wherein the anti-tumor drug comprises anti-cervical cancer drug, anti-breast cancer drug, anti-melanoma drug, anti-lung cancer drug or anti-myeloid cancer drug.

17. A method for preventing and/or treating a tumor, wherein the method comprises: administering the nitrogen-containing polycyclic aromatic compound and the pharmaceutically acceptable salt thereof according to claim 1 as active ingredients to a patient.

18. The method according to claim 17, wherein the tumor comprises at least one of cervical cancer, breast cancer, melanoma, lung cancer and bone marrow cancer.
